(19) United States
(12) Patent Application Publication
(10) Pub. No.: US 2013/0072864 A1
(43) Pub. Date: Mar. 21, 2013

(54) Dual Gravity and Pressure Colon Irrigation Device and Method

(71) Applicant: Delmar Vogel, North Vancouver (CA)

(72) Inventor: Delmar Vogel, North Vancouver (CA)

(21) Appl. No.: 13/235,225

(22) Filed: Sep. 16, 2011

(51) Int. Cl. A61M 3/02; A61M 1/00

(52) U.S. Cl. 604/28; 604/27; 604/31; 604/30

(57) ABSTRACT

The present invention provides a compact colon irrigation device and a method for performing human colon irrigation. The colon irrigation device of the present invention comprises at least a housing, a gravity tank to supply water by gravity, a means for filling the gravity tank with warm water, an extension tube extending vertically from the top of the gravity tank to create a required water head for providing substantially a constant gravity flow of water at a predetermined pressure, a speculum for insertion into rectum of colon for irrigation and extracting matter, a water supply line extending from the gravity tank connected with the said speculum, and a drain line connected with the said speculum. The gravity tank provides water at a pressure suitable for carrying out the colon irrigation safely.

### STATEMENT REGARDING FEDERALLY FUNDED RESEARCH

This invention was not made by any agency of the United States Government nor under any contract with any agency of the United States Government.

### TECHNICAL FIELD OF THE INVENTION

The invention relates to medical equipments and more particularly to colon irrigation devices and a method for performing colon irrigation.

### BACKGROUND OF THE INVENTION

The colon may retain a large amount of matter which accumulates and does not move out of the colon during long periods of time or less mobile matter due to bowel deficiencies. Such conditions of more or less permanent residence of feces material in the colon may lead to toxic effects, inflammation of the lining of the colon, or other serious complications. To avoid such difficulties, colon irrigation is suggested. Colon irrigation is also known by various names such as colon lavaging, colon cleansing, colon washing, and colon hydrotherapy which are herein after referred to as colon irrigation.

Numerous types of systems and device for irrigating and cleansing the colon can be found in the prior art. Prior art systems and devices typically include a fluid (such as water) inlet for introducing water into the apparatus, means for purifying the water such as a filter system, means for controlling the fluid flow and fluid pressure, and a fluid outlet which is in turn connected to a rectal speculum via a tubing.

Tap water or elevated gravity tank water is used for irrigation as source water. Tap water is generally available at a pressure of about 50 to 70 psi. To use tap water, the pressure of tap water is reduced to a predetermined pressure by using a pressure controlling valve, whereas in case of the elevated gravity tank, the gravity tank is adapted above the colon irrigation device at a high position to obtain required pressure with gravity flow of water.

For carrying out irrigation, a speculum secured to flexible hosing is inserted into the anal canal by the attendant and a supply of warm water injected via the speculum into the colon. While the therapist massages the patient's abdomen he observes the pressure of the water within the speculum and patient from a gauge. As the water fills the colon pressure increases. Once the pressure reaches a selected upper level, such as one pound per square inch, above which the patient would experience discomfort, the therapist operates a valve in a drain line that extends from the speculum whereupon the pressure decreases as waste water exits the colon. While drainage occurs the flow of fecal material and water is observed through a sight or view tube so that the therapist may observe the matter being discharged for diagnostic purposes. Once the pressure has dropped substantially, such as to one quarter pound per square inch, the drain valve is closed and the operative cycle repeated until the colon has been well cleansed. However, in the event the attendant or therapist engages in conversation with the patient or becomes otherwise distracted, he or she may fail to observe that the pressure has increased beyond the upper limit. In this case the patient will normally experience discomfort or even pain. A potential for damage to the intestine will also arise. Though gravity flow by using elevated gravity tank solves this problem, the system is cumbersome and requires more floor space.

Currently, various devices have been in use and in patent domain to control the pressure in the colon. However, there is still need for a colon irrigation device, which can reduce potential malfunction of the apparatus in addition to hazards as discussed above and also a method that is less cramping and safer to the patient.

### SUMMARY OF THE INVENTION

The objective of the present invention is to provide a compact colon irrigation device that provides gravity flow and solves at least one of the above described problems or limitations.

Accordingly, in the first aspect, the present invention provides a colon irrigation device comprising a housing, a gravity tank having an inlet, an outlet at the bottom of the gravity tank to supply water by gravity, and an overflow drain, a means for filling the gravity tank with warm water connected to an inlet of the gravity tank, an extension tube extending vertically from the top of the gravity tank to create a required water head for providing a constant gravity flow of water substantially at a predetermined pressure, an overflow drain adapted in connection with the extension tube at a level required for maintaining the required water head in the extension tube, a speculum for insertion into rectum of colon for irrigation and extracting matter, a water supply line extending from the outlet of the gravity tank connected with the said speculum and a drain line connected with the said speculum.

According to the present invention, the means for filling the gravity tank includes a hot supply line and cold water supply line. Advantageously, the means for filling the gravity tank includes a mixer for mixing hot and cold water to supply warm water to the gravity tank.

According to an embodiment of the present invention, the gravity tank can be adapted adjoining to the top panel of housing. The gravity tank is preferably adapted at one of the corners of top panel in the housing. Alternatively, the gravity tank can be adapted over or near the housing.

According to the preferable embodiment of the present invention, the device comprises an automatic shut off valve provided at the means for filling the gravity tank to fill the gravity tank and the extension tube and to maintain the predetermined water head in the extension tube. Alternatively, the automatic shut off valve can be adapted on the overflow drain to control drain so as to maintain the predetermined water head in the extension tube.

According to the present invention, the required water head is maintained in the extension tube to provide water at least at a pressure of about 1 psi to carry out colon irrigation. Accordingly, the required water head is constantly maintained between 20 to 40 inches up in the extension tube.

According to the present invention, a capacity of the gravity tank is between 1-2 liter. Advantageously, the capacity of the gravity tank is 1 liter to make the device compact.

In second aspect, the present invention provides a colon irrigation device, comprising a housing, a gravity tank adapted in the housing, said gravity tank having an inlet and an outlet to supply water by gravity, a means for filling the gravity tank with warm water, an extension tube extending vertically from the top of the gravity tank to create a required water head for providing substantially a constant gravity flow of water at a predetermined pressure, an overflow drain adapted in connection with the extension tube at a level required to maintain the required water head in the extension tube, a speculum for insertion into rectum of colon for irrigation and extracting matter, a water supply line extending from the outlet of the gravity tank connected with the said speculum, a bypass line adapted on the water supply line having a non-return valve operating in proportion to the back pressure developed in the colon to allow gently raising of pressure in the colon and a drain line connected with the said speculum. The non-return valve starts opening at a pressure of about 0.3 psi.

In third aspect, the present invention provides a colon irrigation device, comprising a housing, a first branch line for providing a gravity flow and a second branch line for providing a pressure flow, a three-way valve connected with the first branch line and second branch line a speculum for insertion into rectum of colon for irrigation and extracting matter, a water supply line extending from the three-way valve connected with the said speculum and a drain line connected with the said speculum. The first branch line includes a gravity tank for providing gravity flow, an extension tube extending vertically from the top of the gravity tank to create a required water head for providing substantially a constant gravity flow of water at a predetermined pressure, and an overflow drain for maintaining required water head in the extension tube. The second branch line includes a pressure controlling valve to reduce pressure of water.

In forth aspect, the present invention provides a colon irrigation device, comprising a housing, a first branch fine for providing a gravity flow and a second branch line for providing a pressure flow of water, a three-way valve connected with the first branch line and second branch line, a speculum for insertion into rectum of colon for irrigation and extracting matter, a water supply line extending from the three-way valve connected with the said speculum, a bypass line adapted on the water supply line, having a non-return valve operating in proportion to the back pressure developed in the colon to allow gently raising of pressure in the colon and a drain line connected with the said speculum. The first branch line includes a gravity tank to supply water by gravity, an extension tube extending vertically from the top of the gravity tank to create a required water head for providing substantially a constant gravity flow of water at a predetermined pressure and an overflow drain to maintain the required water head in the extension tube. The second branch line includes a pressure controlling valve to reduce pressure of water.

According to the present invention, the non-return valve start opening at a back pressure of about 0.3 psi and opens full at a predetermined maximum back pressure. Advantageously, the non-return valve set to open full at a back pressure of about between 1 to 5 psi depending on the back pressure in the colon.

In the fifth aspect, the present invention provides a method for performing colon irrigation, comprising steps of using a colon irrigating device of the first aspect and carrying out the colon irrigation by directing gravity water flow from the gravity tank through to the colon for irrigating and extracting waste material therefrom and maintaining a predetermined water head in the extension tube to provide a pressure required to carry out colon irrigation by gravity.

According to an embodiment of the fifth aspect method of the present invention, a step of maintaining a predetermined water head in the extension tube comprises step of controlling water flow from either means for filling the gravity tank or the overflow drain of the gravity tank.

According to further embodiment of the fifth aspect method of the present invention, the method further comprising a step of bypassing the water of the water supply line based on the pressure developed in the colon.

The present invention in sixth aspect provides a method for performing colon irrigation, comprising steps of using a colon irrigating device of the third aspect and carrying out colon irrigation by directing gravity water flow from the gravity tank through to the colon for irrigating and extracting waste material therefrom and maintaining a predetermined water head in the extension tube to provide a pressure required to carry out colon irrigation by gravity. This method also may comprise a step of bypassing the water of the water supply line based on the pressure developed in the colon.

### BRIEF DESCRIPTION OF THE DRAWINGS

The accompanying drawings, which are incorporated into and form a part of the specification, schematically illustrate one or more exemplary embodiments of the present invention and together with the general description given above and detailed description of the preferred embodiments given below serve to explain the principles of the invention:

FIG. 1 shows typical connections of various aspects of a colon irrigation device of the present invention in which:

FIG. 1A shows a typical connection of a colon irrigation device according to first aspect;

FIG. 1B shows a typical connection of a colon irrigation device according to second aspect;

FIG. 1C shows a typical connection of a colon irrigation device according to third aspect; and

FIG. 1D shows a typical connection of a colon irrigation device according to first aspect;

FIG. 2 shows a gravity tank of the colon irrigation device according to present invention;

FIG. 3 shows a various embodiments of the connection between the overflow drain and extension tube according to the present invention through 3A to 3B;

FIG. 4 shows a front perspective view of the exemplary colon irrigation device of the third aspect of the present invention; and

FIG. 5 a perspective view of the colon irrigation device of the third aspect of the present invention revealing internal components arranged in the housing.

### DETAILED DESCRIPTION OF THE PRESENT INVENTION

The present invention provides a compact colon irrigation device and method for performing human colon irrigation.

The colon irrigation device of the present invention generally comprises at least a housing, a gravity tank to supply water by gravity, a means for filling the gravity tank with warm water, an extension tube extending vertically from the top of the gravity tank to create a required water head for providing substantially a constant gravity flow of water at a predetermined pressure, a speculum for insertion into rectum of colon for irrigation and extracting matter, a water supply line extending from the gravity tank connected with the said speculum, and a drain line connected with the said speculum.

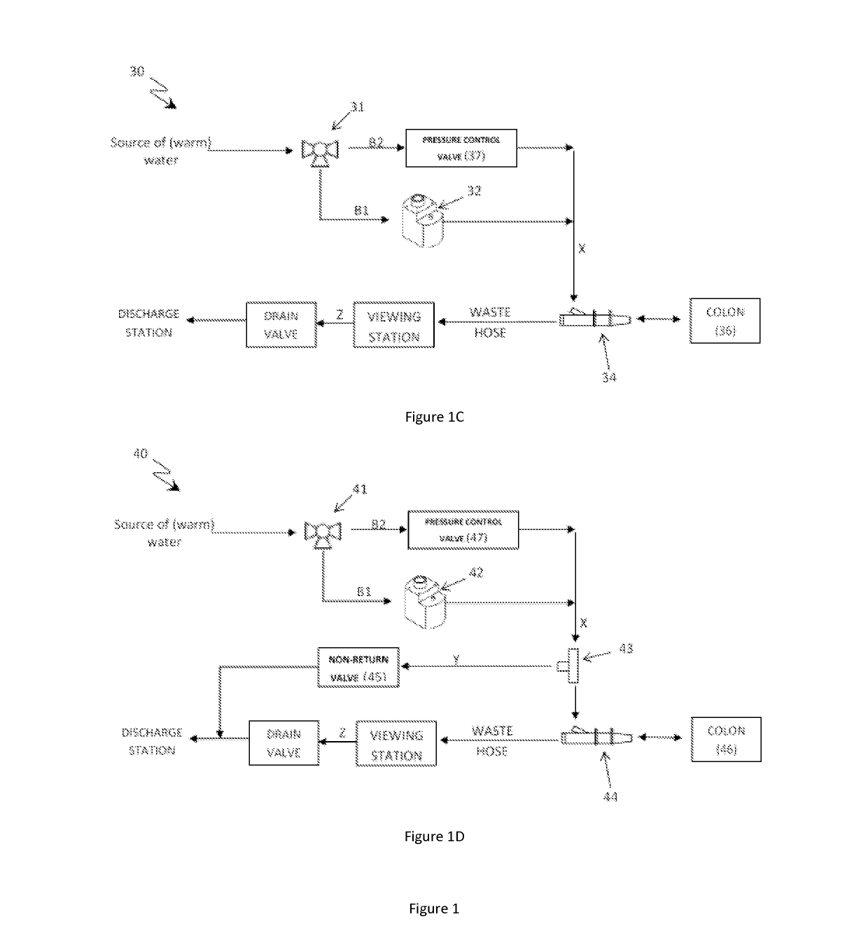

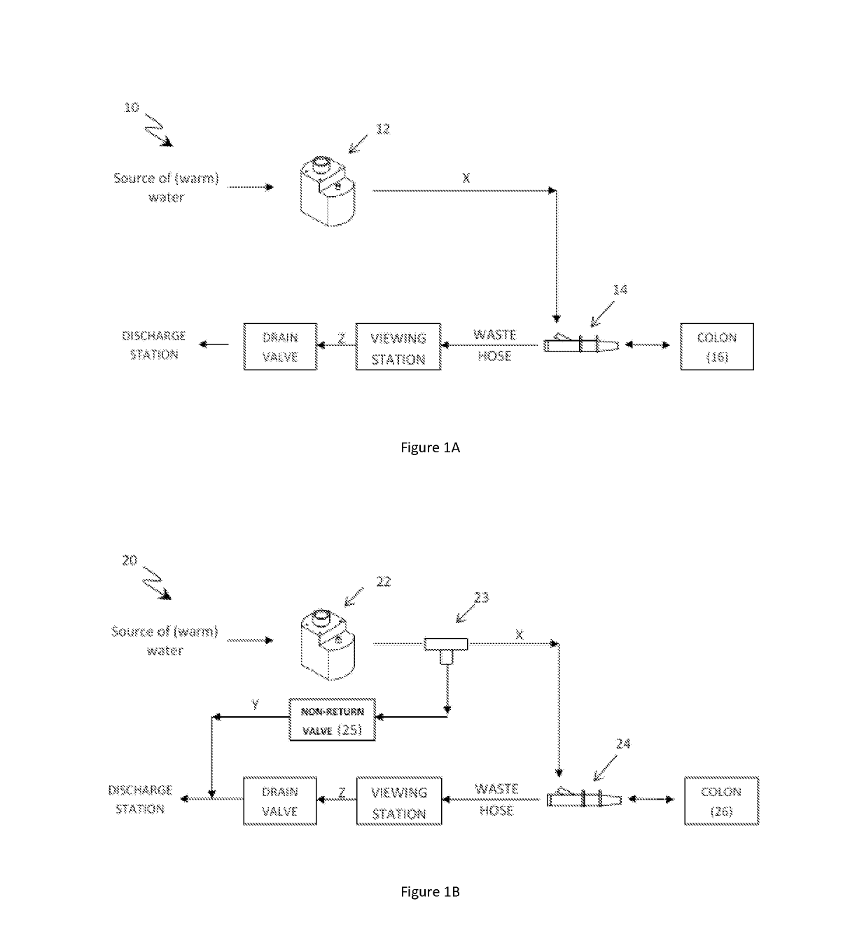

FIG. 1 shows typical connections of various aspects of a colon irrigation device according to the present invention through 1A to 1D.

Referring FIG. 1A shows a typical connection (10) of a colon irrigation device according to first aspect of the invention. The connection shows a source of warm water connected to a gravity tank (12) and a water supplying line (X) extending from the gravity tank (12) to speculum (14) for filling the colon (16). The drain valve provided on the drain line (Z) is opened to drain the colon (16).

Referring FIG. 1B a typical connection (20) of a colon irrigation device according to second aspect of the invention. The connection (20) shows a source of warm water connected to a gravity tank (22) and a water supplying line (X) extending from the gravity tank (22) to a speculum (24) for filling the colon (26) and a bypass line (Y) having a non-return valve (25) of which opening depends upon a pressure developed in a colon (26). The bypass line (Y) extends from a tee (23) provided on the supply line (X) and connected to the drain line. A drain valve provided on the drain line (Z) is opened to drain the colon (26).

Referring FIG. 1C shows a typical connection (30) of a colon irrigation device according to third aspect of the invention. The connection (30) shows the colon irrigation device having two operating modes according to the present invention. A source of warm water after mixer connected to three-way valve (31). First branch (B1) of water extending from the three-way valve (31) is connected to a gravity tank (32) and second branch (B2) extending from the three-way valve (31) is connected to a pressure control valve (37) to operate the colon irrigating device either on gravity mode or pressure mode. The outlet of the gravity tank (32) and the outlet of the pressure control valve (37) are connected at a tee from which a water supply line (X) extends to a speculum (34) for filling colon (36). A drain valve provided on the drain line (Z) is opened to drain the colon (36).

Referring FIG. 1D shows a typical connection (40) of a colon irrigation device according to forth aspect of the invention. The connection (40) shows the colon irrigation device having two operating mode according to the present invention. A source of warm water after mixer connected to three-way valve (41). First branch (B1) of water extending from the three-way valve (41) is connected to a gravity tank (42) and second branch (B2) extending from the three-way valve (41) is connected to a pressure control valve (47) to operate the colon irrigating device either on gravity mode or pressure mode. The outlet of the gravity tank (42) and the outlet of the pressure control valve (47) are connected at a tee (not shown) from which a water supply line (X) extends through a tee (43) to a speculum (44) for filling colon (46). A bypass line is extends from tee (43) and comprises a non-return valve (45) of which opening depends upon a pressure developed in colon (46). A drain valve provided on the drain line (Z) is opened to drain the colon (46).

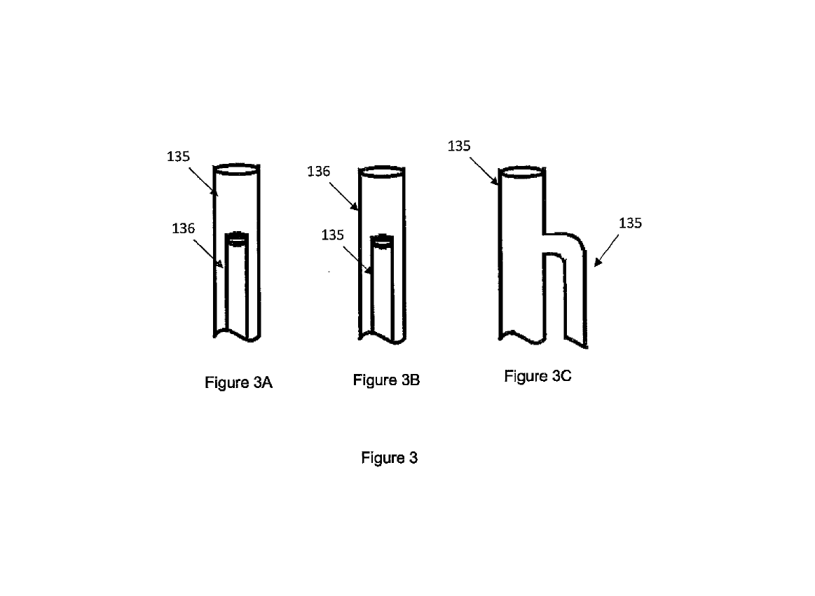

Now, the present invention is described in more detail with respect to gravity tank, supply line, drain line, gauges and other features arranged the colon irrigation device of the present invention with the help of FIGS. 3-5.

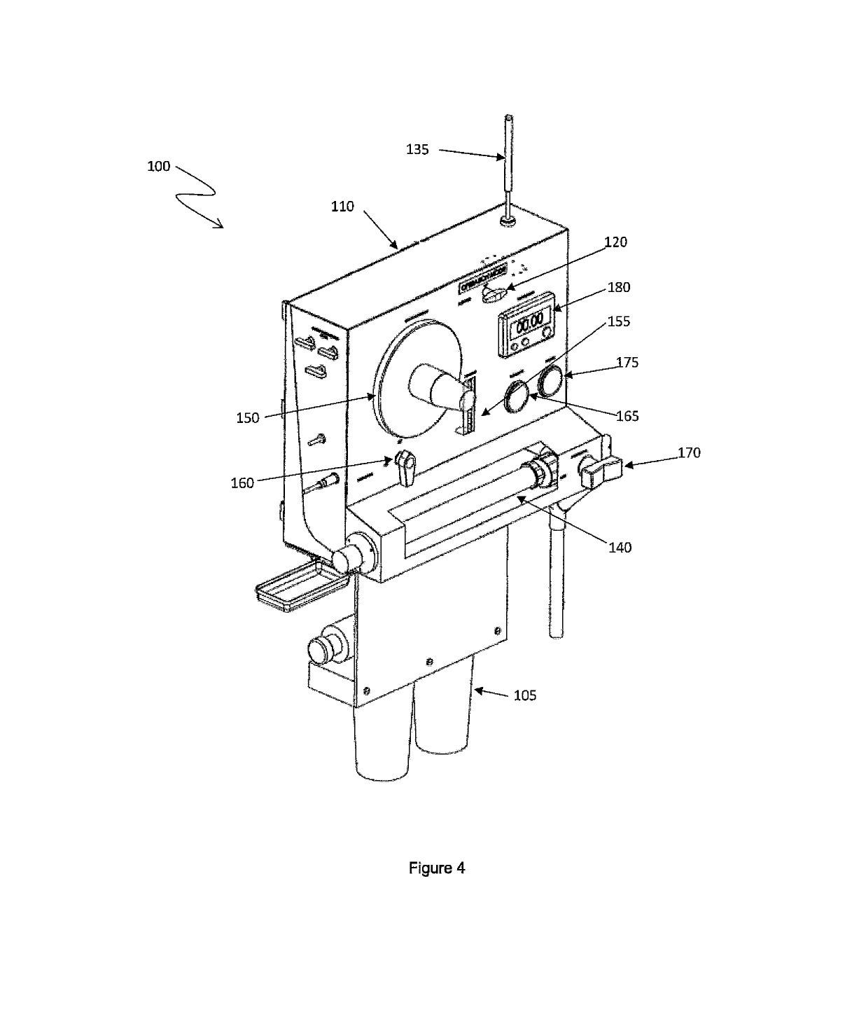

FIG. 4 shows front elevation view of a preferable exemplary embodiment of the colon irrigation device (100) according to the present invention shown in the FIG. 1C. The colon irrigation device (100) includes a housing (110), an operational mode three-way valve (120) for defining a gravity mode or a pressure mode of the device (100) and an extension tube (135) extending above the housing (110) along with regular equipments known in the conventional devices such as a filer unit (105) for filtering water, a window (140) to observe waste water released from the colon, a waste control or drain valve (170), a thermo temperature controller or mixer (150), a flow meter (155), a temperature gauge (165), a pressure gauge (175), a flow control (160) and a treatment timer (180) adapted in defined order on the front panel of the housing (110).

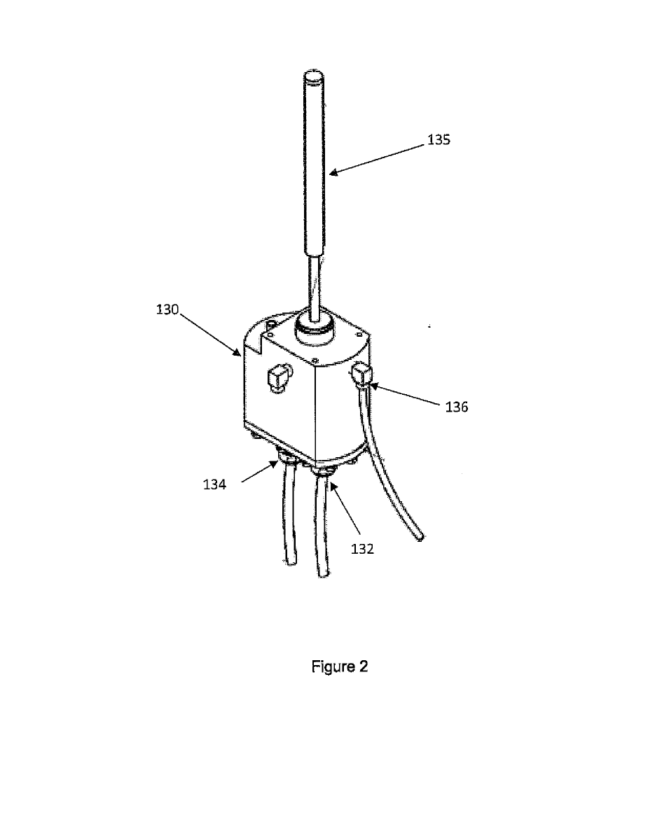

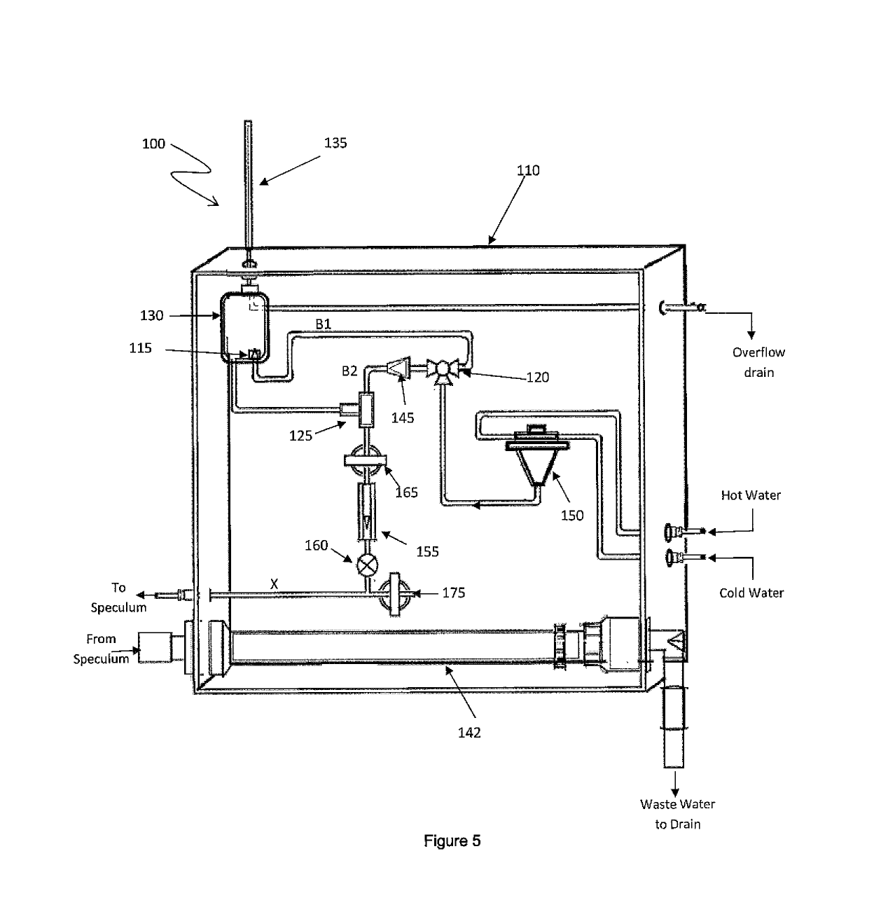

Referring FIG. 5 shows a perspective view revealing inside components of the exemplary colon device shown in FIG. 4. As shown in FIG. 5, cold water and hot water from cold and hot water conduits is mixed in the mixer (150) to obtain warm water at a predetermined temperature. The mixer (150) is connected to the three-way valve (120). The first branch line (B1) extending from the three-way valve (120) comprises an automatic shut off valve (115), the gravity tank (130) and the extension tube (135) of which connection is described below with the help of FIG. 2. The second branch line (B2) extending from the three-way valve (120) comprises the pressure control valve (145). Both the branches (B1, B2) are connected to a tee (125) from which the water supply line (X) extends to the speculum by a known way. The flow meter (155), the temperature (165) and pressure (175) gauges are adapted on the supply line (X) by known ways and are not required to describe in detail.

Referring FIG. 2 shows a gravity tank (130) according to the present invention. The gravity tank (130) comprises an inlet (132) and an outlet (134) at the bottom and an overflow drain (136) at upper level. The extension tube (135) is adapted on the top of the gravity tank (130) to create a required head of water for providing a gravity flow of water at a pressure required for carrying out the colon irrigation. A capacity of the gravity tank is between 1-2 liter. The preferable capacity of the gravity tank is 1 liter to make the device more compact.

FIG. 3 shows various embodiments of the connection between the overflow drain (136) and the extension tube (135) according to the present invention through 3A to 3B.

Referring FIG. 3A shows the inlet of overflow drain (136) extends concentrically within the extension tube (135) to the level required to create a minimum water level in the extension tube (136) for providing the predetermined pressure.

Referring FIG. 3B shows the extension tube extends concentrically within the inlet of overflow drain to the required level thereby maintaining the required level within the extension tube by means of automatic shut-off valve or by allowing overflowing of the extra water from the extension tube. Further, by controlling flow of the water from the overflow drain the water head can be raised up to certain required pressure. This can be done by providing a valve (not shown) to control flow on the overflow drain

Referring FIG. 3C shows an inlet of overflow drain is connected to the extension tube to the level required to maintain the required water head for providing the predetermined pressure.

The shut-off valve (115) is adapted at the inlet of the tank as shown in FIG. 5 to fill the gravity tank (130) and maintain the required head of water in the gravity tank (130). The present invention uses a constant flow of the tap water to provide a gravity flow of water for colon irrigation. Alternatively, the automatic shut-off valve can be adapted on the overflow drain or on both inlet and drain outlet of the gravity tank to maintain preferable level in the extension tube in order to provide gravity flow at a predetermined pressure.

To carry out the colon irrigation by gravity by using a colon irrigation device as shown in FIG. 4, the therapist selects the gravity mode on the colon irrigation device by directing the warm water flow to the tank using a three-way valve. The automatic shut-off valve opens and start filling of the gravity tank. When gravity tank gets full the water, the water level starts raising in the extension tube and when the water level raises up to a level required for providing a required water head to obtain a predetermined pressure, the automatic shut-off valve is activated. Alternatively, or in case of automatic shut-off valve malfunction, draining of water from the over flow drain starts to maintain the required water level in the extension tube. When the water level reaches to a water head required to provide water by gravity at a pressure require carrying out the colon irrigation, the automatic shut off valves starts operating and opens for filling the tank and provides a constant water flow to maintain the said water head in the extension tube. According to the inventor of the present invention, the water head in the extension tube of the gravity tank is required to be maintained in between the 20-40 inches for obtaining a gravity flow of water substantially at least at a pressure of 1 psi since the 1 psi pressure is substantially enough for carrying out colon irrigation and length of the extension tube is directly proportional to the pressure required.

When the water head reaches to a required level, the therapist inserts the speculum into the anal canal of a patient and starts filing warm water into the colon by gravity simultaneously automatic shut off valve starts operating for maintaining required level of water in the extension tube. Advantage of the present invention is that the colon irrigation device provides flow of water by gravity at about 1 psi which fills the colon slowly. This not only avoids a risk of injuring the colon of the patient but also fills the water slowly in the colon without substantially cramping or painful to the patient.

When the colon becomes full of water, a therapist commences a reverse flow by opening the drain valve (170) on the drain line so that water and extracted material flow back through the speculum into the drain line (Z). The waste water flowing from the colon is observed through the optically transparent tube (142) below which by a known way an illumination means (not shown) is arranged.

As shown in FIGS. 1B and 1D, the non-return valve starts opening when back pressure becomes 0.3 psi and opens full if the back pressure reaches to a set pressure which is between 1 to 5 psi. The non-return valve helps to raise the pressure gently in the colon and safeguards from rising of the pressure above predetermined maximum allowable pressure within the colon which makes fill comfort to the patient and also safeguards from damaging of the intestine by not allowing the pressure in colon to increase beyond the upper limit.

A disinfecting or cleaning solution can be mixed in the warm water or waste water before discharging to sewage line for disinfecting or cleaning purpose by known ways.

Thus the present invention has various exceptional features. The device adjusts to the specific conditions of the colon and thereby needs of an individual. Its unique feature senses the applied pressure as it builds in the colon and automatically reduces the flow of water as pressure in the colon increases, thus provides for gentler and longer colon fill cycles. It is therefore an advantage that the present invention considers not only the effective removal of impacted materials but also the safety and comfort of the individual.

According to the present invention, the overflow drain and outlet of the bypass line are connected to the drain line. Alternatively, the overflow drain and the outlet of the bypass line can be connected to other than the drain line.

The foregoing is illustrative of the present invention and is not to be construed as limiting thereof. The electric connection and other known parts are not described herein as the person skilled in the art can easily implement those things in the present invention, Further though a few exemplary embodiments of the present invention have been described, those skilled in the art will readily appreciate that many modifications are possible without materially departing from the novel teachings and advantages of the present invention. Accordingly, all such modifications are intended to be included within the scope of the present invention as defined in the claims. In the claims, means-plus-function clauses are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein:

What is claimed is:

1. A colon irrigation device comprising:
a housing;
a gravity tank having an inlet and an outlet to supply water by gravity;
a means for filling the gravity tank with warm water connected to the inlet of the gravity tank;
an extension tube extending vertically from the top of the gravity tank to create a required water head for providing a constant gravity flow of water substantially at a predetermined pressure;
an overflow drain adapted in connection with the extension tube at a level required for maintaining the required water head in the extension tube;
a speculum for insertion into rectum of colon for irrigation and extracting matter;
a water supply line extending from the outlet of the gravity tank connected with the said speculum; and
a drain line connected with the said speculum.

2. The colon irrigation device of claim 1, wherein the means for filling the gravity tank includes a hot and cold water supply lines.

3. The colon irritation device of claim 1 or 2, wherein the means for filling the gravity tank includes a mixer for mixing hot and cold water to supply warm water to the gravity tank.

4. The colon irrigation device of claim 1, wherein the inlet of overflow drain extends concentrically within the extension tube to the required level.

5. The colon irrigation device of claim 1, wherein the extension tube extends concentrically within the inlet of overflow drain to the required level.

6. The colon irrigation device of claim 1, wherein an inlet of overflow drain is connected to the extension tube to the level required to maintain the required water head.

7. The colon irrigation device of claim 1 wherein the gravity tank is preferably adapted at one of the top corners of housing.

8. The colon irrigation device of claim 1, wherein the device comprises an automatic shut off valve provided at the means for filling the gravity tank to fill the gravity tank and the extension tube and to maintain the predetermined water head in the extension tube.

9. The colon irrigation device of claim 1, wherein the device comprises an automatic shut off valve adapted on the overflow drain to control drain so as to maintain the predetermined water head in the extension tube.

10. The colon irrigation device of claim 1, wherein the required water head is maintained in the extension tube to provide water at least at 1 psi.

11. The colon irrigation device of claim 1, wherein the required water head is constantly maintained between 20 to 40 inches up in the extension tube

12. The colon irrigation device of claim 1, wherein a capacity of the gravity tank is between 1-2 liter.

13. The colon irrigation device of claim 1, wherein the capacity of the gravity tank is preferably 1 liter.

14. A colon irrigation device, comprising:
a housing;
a gravity tank adapted in the housing, said gravity tank having an inlet, and an outlet to supply water by gravity;
a means for filling the gravity tank with warm water;
an extension tube extending vertically from the top of the gravity tank to create a required water head for providing substantially a constant gravity flow of water at a predetermined pressure;
an overflow drain adapted in connection with the extension tube at a level required to maintain the required water head in the extension tube;
a speculum for insertion into rectum of colon for irrigation and extracting matter;
a water supply line extending from the outlet of the gravity tank connected with the said speculum;
a bypass line adapted on the water supply line having a non-return valve operating in proportion to the back pressure developed in the colon to allow gently raising of pressure in the colon; and
a drain line connected with the said speculum.

15. The colon irrigation device of claim 14, wherein the non-return valve starts opening at a pressure of about 0.3 psi.

16. A colon irrigation device, comprising:
a housing;
a first branch including a gravity tank for providing gravity flow an extension tube extending vertically from the top of the gravity tank to create a required water head for providing substantially a constant gravity flow of water at a predetermined pressure and an overflow drain for maintaining required water head in the extension tube;
a second branch line including a pressure controlling valve to reduce pressure of water;
a three-way valve connected with the first branch line and second branch line;
a speculum for insertion into rectum of colon for irrigation and extracting matter;
a water supply line extending from the three-way valve connected with the said speculum; and
a drain line connected with the said speculum.

17. A colon irrigation device, comprising:
a housing having a front panel, a back panel, a top panel, a bottom panel and opposing side panel;
a first branch including a gravity tank for providing gravity flow, an extension tube extending vertically from the top of the gravity tank to create a required water head for providing substantially a constant gravity flow of water at a predetermined pressure and an overflow drain for maintaining required water head in the extension tube;
a second branch line including a pressure controlling valve to reduce pressure of water;
a three-way valve connected with the first branch line and second branch line;
a speculum for insertion into rectum of colon for irrigation and extracting matter;
a water supply line extending from the three-way valve connected with the said speculum;
a bypass line adapted on the water supply line, having a non-return valve operating in proportion to the back pressure developed in the colon to allow gently raising of pressure in the colon; and
a drain line connected with the said speculum.

18. The colon irrigation device of claim 17, wherein the non-return valve starts opening at a pressure of about 0.3 psi.

19. The colon irrigation device of claim 17, wherein the non-return valve opens full at a predetermined maximum back pressure.

20. The colon irrigation device of claim 17, wherein the non-return valve can be set to open full at a pressure of about between 1 to 5 psi.

21. A method for performing colon irrigation, said method comprising steps of
i) using a colon irrigating device comprising of
a. a housing,
b. a gravity tank adapted within the said housing, said gravity tank having a means for filling the gravity tank, an outlet at the bottom of the gravity tank to supply warm water by gravity;
c. an extension tube extending vertically from the top of the gravity tank to create a required water head for providing substantially a constant gravity flow of water at a predetermined pressure
d. an overflow drain adapted in connection with the extension tube at a level required for maintaining the required water head in the extension tube,
e. a speculum for insertion into rectum of colon for irrigation and extracting matter,
f. a water supply line extending from the gravity tank connected with the said speculum, and
g. a drain line connected with the said speculum;
ii) carrying out colon irrigation by directing gravity water flow from the gravity tank through to the colon for irrigating and extracting waste material therefrom and maintaining a predetermined water head in the extension tube to provide a pressure required to carry out colon irrigation by gravity.

22. The method for performing colon irrigation of claim 21, wherein a step of maintaining a predetermined water head in the extension tube comprises step of controlling waterflow from either means for filling the gravity tank or the overflow drain of the gravity tank.

23. The method for performing colon irrigation of claim 21 further comprising a step of bypassing the water of the water supply line base on the pressure developed in the colon.

24. A method for performing colon irrigation, said method comprising steps of
i) using a colon irrigating device comprising of
a. a housing,
b. a first branch including a gravity tank for providing gravity flow, an extension tube extending vertically from the top of the gravity tank to create a required water head for providing substantially a constant gravity flow of water at a predetermined pressure and an overflow drain for maintaining required water head in the extension tube,
c. a second branch line including a pressure controlling valve to reduce pressure of water,
d. a three-way valve connected with the first branch line and second branch line;
e. a speculum for insertion into rectum of colon for irrigation and extracting matter,
f. a water supply line extending from the three-way valve connected with the said speculum, and
g. a drain line connected with the said speculum; and
ii) carrying out colon irrigation by directing gravity water flow from the gravity tank through to the colon for irrigating and extracting waste material therefrom and maintaining a predetermined water head in the extension tube to provide a pressure required to carry out colon irrigation by gravity.

25. The method for performing colon irrigation of claim 24 further comprising a step of bypassing the water of the water supply line base on the pressure developed in the colon.
